(19) United States
(12) Patent Application Publication
(10) Pub. No.: US 2021/0312594 A1
(43) Pub. Date: Oct. 7, 2021

(54) ULTRASONIC IMAGING APPARATUS AND IMAGE PROCESSING APPARATUS

(71) Applicant: Hitachi, Ltd., Tokyo (JP)

(72) Inventors: Kazuhiro YAMANAKA, Tokyo (JP); Teiichiro IKEDA, Tokyo (JP); Nobuhiko FUJII, Tokyo (JP); Misaki HIROSHIMA, Tokyo (JP)

(21) Appl. No.: 17/217,028

(22) Filed: Mar. 30, 2021

(30) Foreign Application Priority Data
Apr. 1, 2020 (JP) 2020-066146

(51) Int. Cl. G06T 5/00; G06T 5/20; A61B 8/14; A61B 8/00; A61B 8/08; G16H 50/20; G16H 30/40

(52) U.S. Cl. CPC G06T 5/002; G06T 5/20; A61B 8/14; A61B 8/54; G06T 2207/20081; A61B 8/5207; G16H 50/20; G16H 30/40; G06T 2207/10132; A61B 8/5269

(57) ABSTRACT

An ultrasonic imaging apparatus is capable of removing noise for each tissue from a received signal of a living tissue in which a plurality of tissues are intricately intertwined, and suppressing artifacts even at a tissue boundary. An evaluator receives one or more time-series ultrasonic signals obtained by receiving an ultrasonic wave from a subject by an ultrasonic probe after transmitting an ultrasonic wave, and generates a filter control signal for setting a characteristic value of a filter for removing noise contained in the ultrasonic signal, for a time-series direction of the ultrasonic signal. A filter processor removes the noise by processing the ultrasonic signal of the corresponding signal characteristic value by the filter having the characteristic value set based on the filter control signal. A smoothing processor that smooths distribution of the filter control signal is disposed between the evaluator and the filter processor.

### CROSS-REFERENCE TO RELATED APPLICATION

The present application claims priority from Japanese application JP2020-066146, filed on Apr. 1, 2020, the contents of which is hereby incorporated by reference into this application.

### BACKGROUND OF THE INVENTION

### Field of the Invention

The present invention relates to an ultrasonic imaging apparatus, and particularly to an apparatus capable of generating an image with reduced noise even in a complex tissue such as a living body.

### Background Art

An apparatus is known in which an ultrasonic wave is transmitted from an ultrasonic probe to an object, the ultrasonic wave scattered and reflected inside the object is received again by the ultrasonic probe, and the image in the object is generated from an obtained received signal, and an apparatus for inspecting the presence or absence of flaws in the object from the obtained received signal is known. In these apparatuses, if the obtained received signal contains noise, there may be problems such as deterioration of image quality of the generated image and erroneous determination that a non-flawed portion in the object is a flaw.

Therefore, for example, Patent Literature 1 proposes an ultrasonic flaw detection apparatus that calculates a feature amount of the received signal by performing wavelet transform on the received signal, and distinguishes between an echo reception signal from the flaw and an echo reception signal at a welded portion that is not from the flaw based on a difference in distribution of equiphase surfaces.

Patent Literature 2 discloses that an estimated value of an electromagnetic noise signal contained in the received signal is determined by analyzing the received signal, and the received signal is modified based on the determined estimated value. Specifically, in Patent Literature 2, the estimated value of an electromagnetic noise component signal is determined by utilizing the fact that phases of signal components from an imaging target differ depending on a channel but phases of the electromagnetic noise components are the same, in the received signal that reaches an ultrasonic element in an edge region of the ultrasonic probe, and by averaging the received signals of the ultrasonic element in the edge region. The estimated value of the determined electromagnetic noise component signal is subtracted from the received signal of each channel, and then a reception beam is formed. Thus, in the technique of Patent Literature 2, an ultrasonic image with reduced electromagnetic noise signal is generated.

### CITATION LIST

### Patent Literature

Patent Literature 1: JP-A-2001-165912

Patent Literature 2: JP-A-2012-055692

### SUMMARY OF THE INVENTION

### Technical Problem

A living body includes various tissues intricately intertwined, and the tissues have a characteristic that scattering characteristics and reflection characteristics of the ultrasonic wave are significantly different. Therefore, the received signals obtained by transmitting the ultrasonic wave to the living body have different signal strengths of the received signals derived from the tissues depending on a type of tissue and a shape of a boundary at a point where the transmitted ultrasonic wave is scattered or reflected, and characteristics such as noise amplitude and frequency are also different. For example, when the noise of the received signal is removed for each reception scanning line, characteristics of the noise are different for a plurality of tissues arranged in a depth direction of the reception scanning line, and in order to properly remove the noise, it is necessary to set different denoising parameter values for each tissue in the depth direction.

Therefore, for the living body containing various tissues, when trying to correct the received signal by calculating or estimating the feature amount of the received signal and the electromagnetic noise contained in the received signal as in the inventions of Patent Literature 1 and Patent Literature 2, the feature amount and the noise differ greatly depending on the type of tissue containing the point where the ultrasonic wave is scattered and reflected, and a correction amount of the received signal also differs greatly.

Therefore, when targeting the living body, it is necessary to properly remove the noise at each position in a scanning direction and a depth direction of a transmitted beam.

However, since the technique of Patent Literature 1 is the ultrasonic flaw detection apparatus, it distinguishes between the flaw and the welded portion in the same material, and thus the denoising parameter values cannot be set for each living tissue in which the tissues are intertwined.

The technique of Patent Literature 2 is a method in which the estimated value of the electromagnetic noise component signal is determined by averaging the received signals of each ultrasonic element in the edge region of the ultrasonic probe, the estimated value is subtracted from the received signal of each channel, and then the reception beam is formed. Therefore, it is not clear to what extent the noise of various frequency components included in the received signal can be removed by the technique of Patent Literature 2.

Therefore, in order to properly remove noise for each tissue arranged on the reception scanning line, it is necessary to set an appropriate value for the denoising parameter for each tissue. In this case, since the denoising parameter changes significantly at a tissue boundary, it is necessary to suppress artifacts and the like that may occur due to the parameter change.

An object of the present invention is to provide an ultrasonic imaging apparatus capable of properly removing noise for each tissue from the received signal of the living tissue in which the tissues are intricately intertwined, and suppressing the artifacts even at the tissue boundary.

### Means for Solving the Problems

In order to achieve the above object, an ultrasonic imaging apparatus of the present invention includes: an evaluator that generates a filter control signal, in which the evaluator receives one or more time-series ultrasonic signals obtained by receiving an ultrasonic wave from a subject by an ultrasonic probe after transmitting an ultrasonic wave from the ultrasonic probe to the subject, and generates the filter control signal for setting a characteristic value of a filter for removing noise contained in the ultrasonic signal, for a time-series direction of the ultrasonic signal or for a predetermined direction or region set in a signal space of two or more dimensions in which a plurality of the ultrasonic signals are arranged; a filter processor that removes the noise by processing the ultrasonic signal of the corresponding signal characteristic value by the filter having the characteristic value set based on the filter control signal; and an image processor that generates an image using the ultrasonic signal from which the noise has been removed by the filter processor. A smoothing processor that smoothes distribution of the filter control signal in the direction or region and/or distribution of the ultrasonic signals after filter processing is disposed between the evaluator and the filter processor and/or between the filter processor and the image processor.

### Advantage of the Invention

According to the present invention, it is possible to properly remove the noise for each tissue from the received signal of the living tissue in which the tissues are intricately intertwined, and to suppress the artifacts even at the tissue boundary.

### BRIEF DESCRIPTION OF THE DRAWINGS

FIG. 1 is a perspective view of an ultrasonic imaging apparatus of Embodiment 1 of the present invention;

FIG. 2 is a block diagram illustrating a configuration of the ultrasonic imaging apparatus of Embodiment 1;

FIG. 3A is a block diagram of a main part of the ultrasonic imaging apparatus of Embodiment 1, and FIGS. 3B to 3D are explanatory views illustrating processing of an evaluator 107 and a smoothing processor 108-1;

FIG. 4 is a flowchart illustrating an operation of the ultrasonic imaging apparatus of Embodiment 1;

FIG. 5A is a block diagram of the main part of the ultrasonic imaging apparatus of Embodiment 2, and FIGS. 5B to 5D are explanatory views illustrating processing of the evaluator 107 and a smoothing processor 108-2;

FIG. 6A is an explanatory diagram illustrating weights used by the smoothing processor 108-2 of Embodiment 2, and FIG. 6B is a block diagram illustrating the processing of the smoothing processor 108-2;

FIG. 7 is a block diagram of the main part of the ultrasonic imaging apparatus of Embodiment 3;

FIG. 8 is a block diagram of the main part of the ultrasonic imaging apparatus of Embodiment 4;

FIG. 9A is a flowchart illustrating a prescan operation of the ultrasonic imaging apparatus of Embodiment 5, and FIG. 9B is a table showing a transmission beam of a prescan sequence;

FIG. 10A is a block diagram of the main part of the ultrasonic imaging apparatus of Embodiment 6, and FIG. 10B is a flowchart illustrating a prescan operation of the ultrasonic imaging apparatus of Embodiment 6;

FIGS. 11A and 11B are block diagrams of the main part of the ultrasonic imaging apparatus of Embodiment 7; and

FIG. 12 is a block diagram of the main part of the ultrasonic imaging apparatus of Embodiment 8.

### DESCRIPTION OF THE PREFERRED EMBODIMENTS

Hereinafter, an ultrasonic imaging apparatus of an embodiment of the present invention will be described with reference to the drawings.

### Embodiment 1

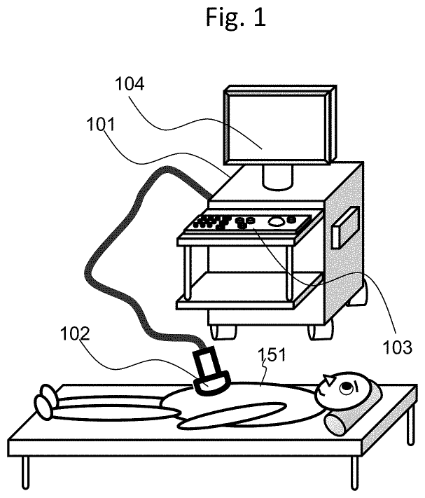

A configuration of an ultrasonic imaging apparatus 100 of Embodiment 1 will be described with reference to FIGS. 1 to 3D.

The ultrasonic imaging apparatus 100 of the present embodiment includes an ultrasonic imaging apparatus main body 101, and an ultrasonic probe 102, a display device 104, and an operation unit 93 are connected to the ultrasonic imaging apparatus main body 101. The ultrasonic probe 102 includes a plurality of ultrasonic elements arranged.

The ultrasonic imaging apparatus main body 101 is configured to include a transmission beamformer (transmission controller) 91, a reception beamformer 105, an evaluator 107, a smoothing processor 108-1, a filter processor 106, and a smoothing processor 108-2, an image processor 109, and a controller 92.

The transmission beamformer 91 outputs a transmission signal to the ultrasonic element of the ultrasonic probe 102 to control transmission.

The reception beamformer 105 delays received signals respectively received by the ultrasonic elements of the ultrasonic probe 102 so as to focus on a plurality of points on a predetermined reception scanning line in a depth direction thereof and then adds them, so that a time-series ultrasonic signal (reception beam) 201 is generated.

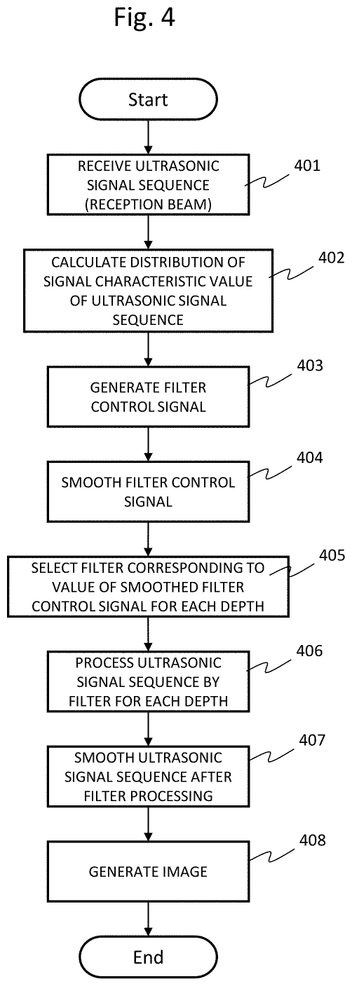

Operations of the evaluator 107, the smoothing processor 108-1, the filter processor 106, the smoothing processor 108-2, and the image processor 109 will be described with reference to the flow of FIG. 4.

The evaluator 107, the smoothing processor 108-1, the filter processor 106, the smoothing processor 108-2, and the image processor 109 include processors such as a CPU (Central Processing Unit) and a GPU (Graphics Processing Unit), and memory. The CPU realizes these functions by software by reading and executing a program stored in the memory. Note that it is also possible to realize a part or all of the evaluator 107, the smoothing processor 108-1, the filter processor 106, the smoothing processor 108-2, and the image processor 109 by hardware. For example, a signal processor 7 may be configured by using a custom IC such as an ASIC (Application Specific Integrated Circuit) or a programmable IC such as an FPGA (Field-Programmable Gate Array), and a circuit may be designed to realize functions of each part of the signal processor 7.

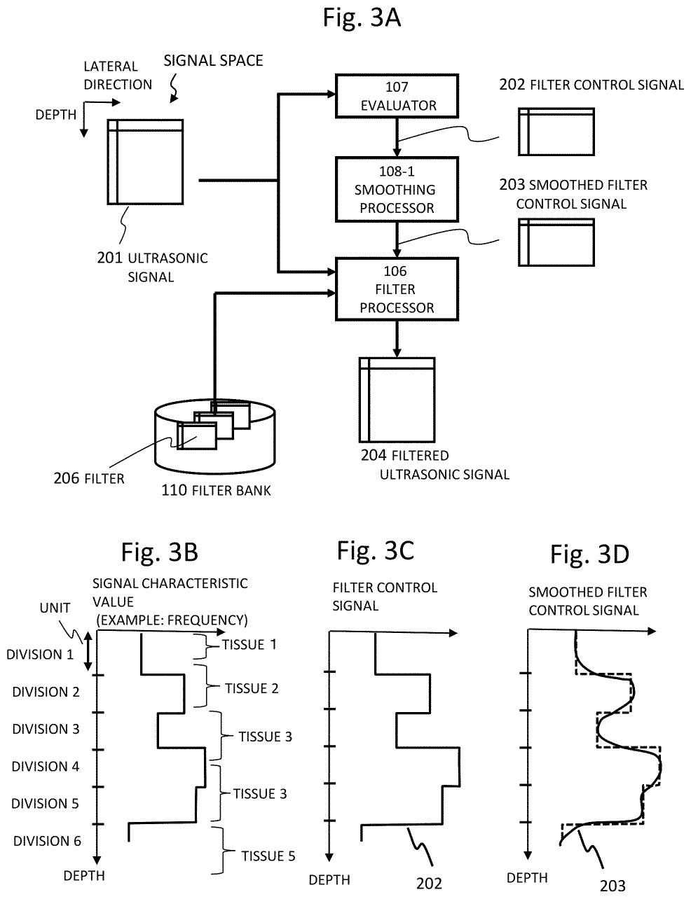

The evaluator 107 receives one or more ultrasonic signals 201 from the reception beamformer 105 (Step 401). Then, the evaluator 107 divides a time direction (depth direction) of the ultrasonic signal 201 by a predetermined unit (length), and calculates a signal characteristic value of the ultrasonic signal 201 for each division to determine distribution of signal characteristic values as illustrated in FIG. 3B (Step 402). Examples of the signal characteristic values include signal strength and frequency.

At this time, since a subject 151 is a living body and includes a plurality of tissues intricately intertwined, the signal characteristic value (for example, frequency) of the ultrasonic signal 201 changes significantly at a boundary of divisions near a boundary of different tissues. For example, in the example of FIG. 3B, there are five tissues on the reception scanning line, and the signal characteristic value (for example, frequency) of the ultrasonic signal 201 is significantly different in each tissue, and thus the signal characteristic value changes significantly at the boundary of divisions.

The evaluator 107 generates a filter control signal for setting a characteristic value of a filter for removing noise contained in the ultrasonic signal according to the signal characteristic value determined in Step 402, in the direction (depth direction) of the ultrasonic signal 201 as in FIG. 3C (Step 403). As the characteristic value of the filter for removing the noise contained in the ultrasonic signal 201, for example, intensity of filter processing or frequency characteristics (center frequency of bandpass filter, window width or the like) of the filter processing can be used.

At this time, since the signal characteristic values (for example, frequencies) of the ultrasonic signal 201 are significantly different along the reception scanning line, a filter control signal for selecting filters having significantly different characteristic values is set as in FIG. 3C.

When the filter selected by the filter control signal is directly applied to the ultrasonic signal (reception beam), filters having significantly different filter characteristic values are applied to the ultrasonic signal 201 on both sides of a tissue boundary. In this case, the ultrasonic signal 201 after the filter processing is discontinuous at the tissue boundary, which may cause artifacts in a generated image.

Therefore, in the present embodiment, the smoothing processor 108-1 is disposed between the evaluator 107 and the filter processor 106, and a filter control signal 202 is smoothed to be changed smoothly as in FIG. 3D in the smoothing processor 108-1 (Step 404).

The filter processor 106 receives a filter control signal 203 after smoothing from the smoothing processor 108 as in FIG. 3D, and selects a filter having a filter characteristic value (weight) corresponding to a value of the filter control signal from filters 206 pre-stored in a filter bank 110 connected to the filter processor 106 (Step 405).

Subsequently, the filter processor 107 processes the ultrasonic signal 201 with the filter selected for each depth (Step 406).

The smoothing processor 108-2 further smoothes a ultrasonic signal 204 filtered in Step 406, if necessary (Step 407).

The image processor 109 generates an ultrasonic image 1206, for example, by arranging the ultrasonic signals 205 smoothed in Step 407 in a lateral direction, and displays it on the display device 104 (Step 408).

According to the ultrasonic imaging apparatus of the present embodiment, the evaluator 107 generates an adaptive filter control signal in response to the ultrasonic signal 201, and moreover, as illustrated in FIG. 3D, discontinuity of the filter control signal at the tissue boundary is corrected to a smooth change by smoothing process. This makes it possible to suppress the artifacts caused by discontinuity in the ultrasonic signal after the filter processing and provide an image to be easily diagnosed. In addition, the noise can be properly removed for each tissue from the received signal of the living tissue in which the tissues are intricately intertwined.

Note that in the above-described embodiment, it may be configured so that only one of the smoothing processor 108-1 and the smoothing processor 108-2 is provided.

As illustrated in FIG. 3A, the ultrasonic signals (reception beams) 201 on the reception scanning line may be arranged in the lateral direction and/or a frame direction to generate a signal space of two or more dimensions. In this case, the evaluator 107 divides a predetermined direction or region in the signal space by a unit for calculating the distribution of the signal characteristic values of the ultrasonic signals (a predetermined length in the lateral direction, a predetermined time length in the frame direction, a predetermined area or volume in the space of two or more dimensions), determines the characteristic value of the filter for removing the noise contained in the ultrasonic signal according to the signal characteristic value for each division, and generates the filter control signal for each division. For example, the predetermined direction can be set to a direction (frame direction) in which frames obtained at the same position of the subject at different times are arranged.

This makes it possible to suppress the discontinuity in the ultrasonic signal after the filter processing due to the discontinuity of the filter characteristics, and to provide the image to be easily diagnosed, by smoothing the filter control signal at the boundary of the division even when the division divided by a unit for calculating the signal characteristics of the ultrasonic signal is set not only in the depth direction but also in an arbitrary region or the frame direction.

In the present embodiment, an interpolation process may be performed instead of or in addition to the smoothing process.

Note that in the present embodiment, the signal characteristic value is determined for each division divided by the unit, but it may be configured so that the signal characteristic values are determined substantially continuously in the predetermined direction or region by making the unit extremely small, the filter control signals are generated substantially continuously based on the signal characteristic values, and the generated filter control signals and/or the ultrasonic signals after the filter processing are smoothed.

In the above-described Embodiment 1, the evaluator 107 is configured to calculate the signal characteristics of the ultrasonic signal 201 and calculate the filter control signal from the signal characteristics by software, but the evaluator 107 can also be constructed by a learning model of machine learning or deep learning (for example, CNN (Convolution Neural Network)). This learning model is trained using teacher data that uses a large number of ultrasonic signals determined in advance as input data and the corresponding appropriate filter control signals as output data, and weighting of nodes in the CNN is set. Thus, by inputting the ultrasonic signal 201 into the trained learning model, the appropriate filter control signal can be output, so that the evaluator 107 can be constructed by the learning model. In this case, the filter control signal can be obtained without calculating the signal characteristics of the ultrasonic signal 201.

When the ultrasonic signal 201 is processed by the filter 206, the filter processor 106 can be configured to weight the parameter value of the filter 206 and perform a convolution operation with the ultrasonic signal 201. In this case, the filter processor 106 may be configured to further include a weight setting unit for generating weights for weighting the parameter values of the filter 206 by the learning model of machine learning or deep learning. The learning model is trained using as teacher data a combination of a large number of ultrasonic signals and filter control signals determined in advance and the weights when the noise can be properly removed. Thus, an appropriate weight can be generated by the learning model of the weight setting unit. The filter processor 106 can obtain the ultrasonic signal from which noise has been removed by weighting the parameter value of the filter 206 and performing the convolution operation with the ultrasonic signal 201.

### Embodiment 2

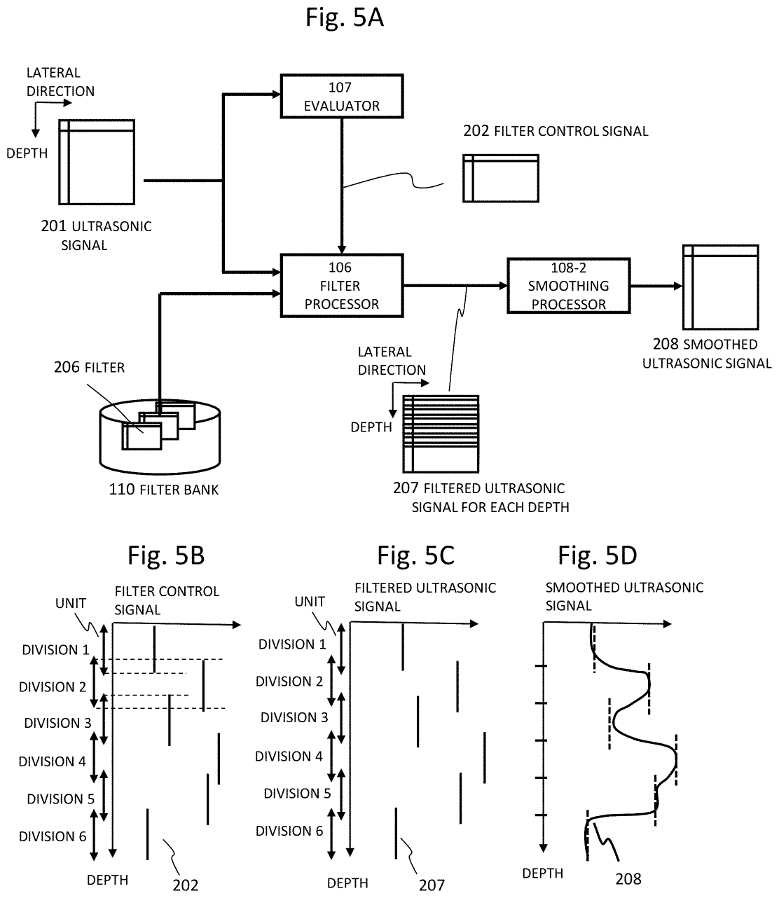

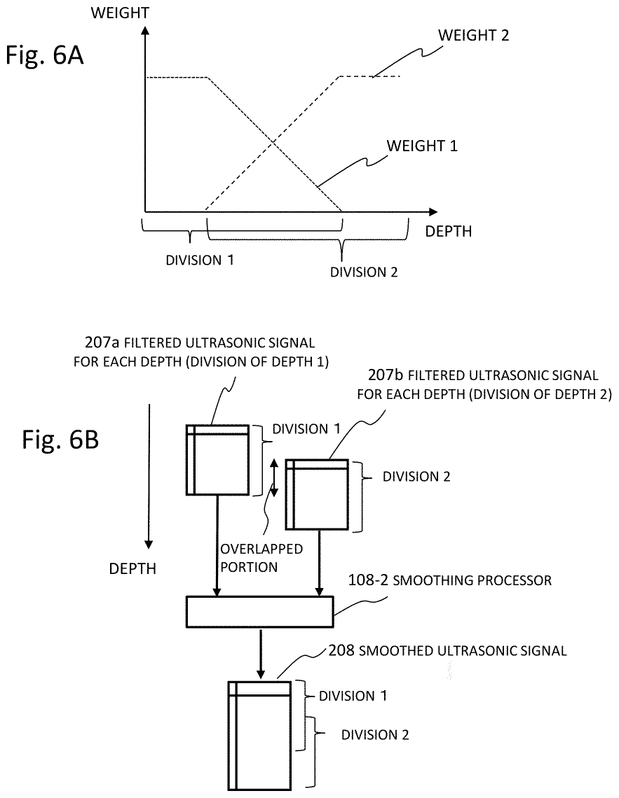

The ultrasonic imaging apparatus of Embodiment 2 will be described with reference to FIGS. 5A to 5D and FIGS. 6A and 6B.

As illustrated in FIG. 5A, the configuration of the ultrasonic imaging apparatus of Embodiment 2 is the same as that of Embodiment 1, but is different from that of Embodiment 1 in that when the evaluator 107 sets divisions in which the signal characteristic value and the filter control signal 202 are generated, it sets the divisions so that adjacent divisions partially overlap each other as illustrated in FIG. 5B.

The filter processor 106 selects the filter 206 based on the filter control signal 202 for each division, and processes the ultrasonic signal of the corresponding division to obtain an ultrasonic signal 207 after the filter processing (hereinafter referred to as a filtered ultrasonic signal) as in FIGS. 5A and 5C. Since the filtered ultrasonic signal 207 is obtained for each division, two signals having different values processed by different filters 206 are obtained at a portion where the adjacent divisions overlap each other.

The smoothing processor 108-2 smoothes two filtered ultrasonic signals 207 in portions where the divisions overlap each other, by weighting them with different weights depending on a predetermined depth as illustrated in FIGS. 6A and 6B and adding them, to obtain a smoothed ultrasonic signal 208.

The image processor 109 generates the ultrasonic image 1206 by using the smoothed ultrasonic signal 208.

Similar to Embodiment 1, the ultrasonic imaging apparatus of Embodiment 2 can suppress the discontinuity in the filtered ultrasonic signal due to the discontinuity of the filter characteristics, and can provide the image to be easily diagnosed.

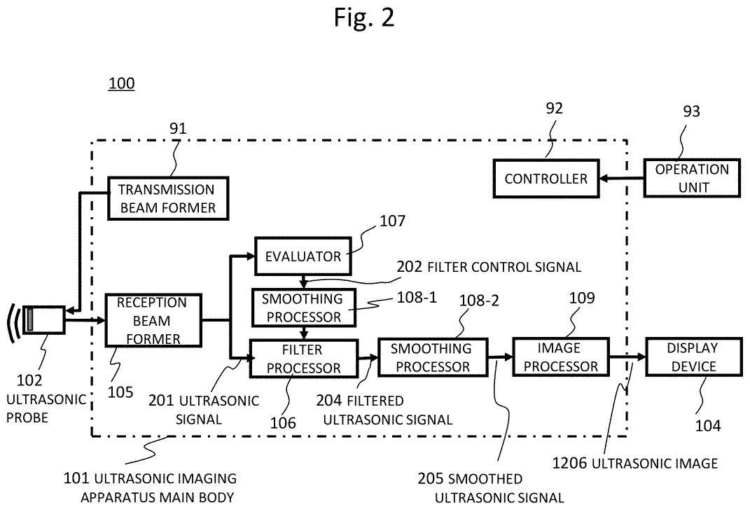

Note that in the above description, the ultrasonic signals 207 after the filter processing are weighted and added by the smoothing processor 108-2, but the filter control signals of FIG. 5B may be weighted and added by the smoothing processor 108-1 of FIGS. 2 and 3.

### Embodiment 3

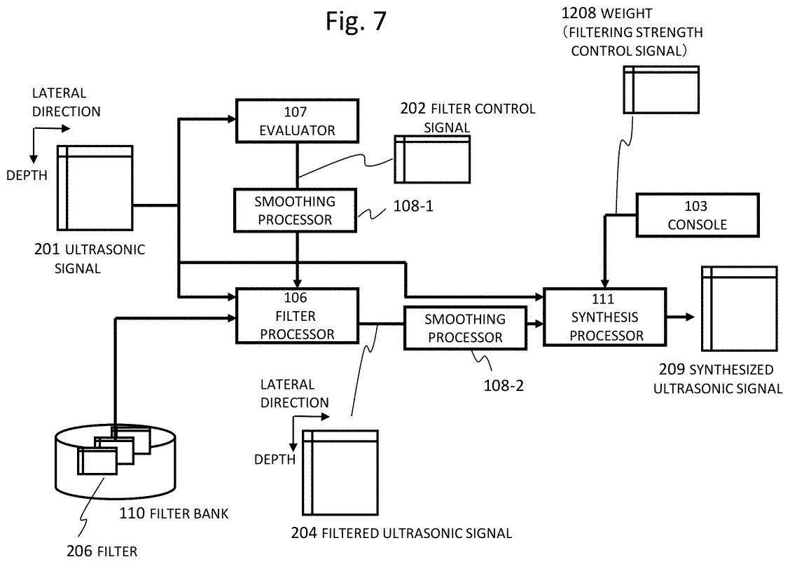

The ultrasonic imaging apparatus of Embodiment 3 will be described with reference to FIG. 7.

The ultrasonic imaging apparatus of Embodiment 3 includes a synthesis processor 111 that weights and synthesizes the ultrasonic signal 201 and the filtered ultrasonic signal 204.

The image processor 109 generates the image using the ultrasonic signal after synthesis by the synthesis processor 111.

A console 103 is connected to the synthesis processor 111, and a user can input weights 1208 when synthesizing signals from a user interface such as a touch panel or a mouse provided in the console 103.

Thus, similar to Embodiment 1, the evaluator 107 generates the adaptive filter control signal in response to the ultrasonic signal, but intensity of the filtered ultrasonic signal 204 reflected in the displayed image can be adjusted according to the user's preference.

Since other configurations are the same as those in Embodiment 1, description thereof will be omitted.

### Embodiment 4

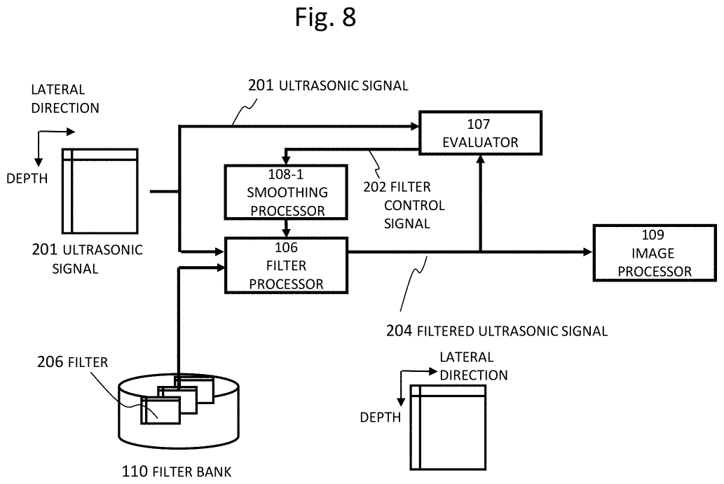

The ultrasonic imaging apparatus of Embodiment 4 will be described with reference to FIG. 8.

The ultrasonic imaging apparatus of Embodiment 4 has the same configuration as that of Embodiment 1, but is different from Embodiment 1 in that the filtered ultrasonic signal 204 is fed back to the evaluator 107.

The evaluator 107 determines a difference or correlation characteristics between the ultrasonic signal 201 and the filtered ultrasonic signal 204 that has been fed back, or a difference in frequency characteristics or correlation characteristics between the ultrasonic signal 201 and the filtered ultrasonic signal 204, and generates an evaluation value for determining validity of the filter processing of the filter processor 106 based on the determined difference or the correlation characteristics. Determining the validity of the filter processing means determining whether the noise is effectively removed from the ultrasonic signal 201 and whether the ultrasonic signal is not removed more than the noise. The evaluator 107 reflects the generated evaluation value in generation of the filter control signal 202, and controls feedback so that the evaluation value is increased.

The smoothing processor 108-1 generates a smoothed filter control signal from the filter control signal used when processing the ultrasonic signals of a plurality of frames up to just before. For example, the smoothed filter control signal for a current frame is generated by calculating an average value of the filter control signals of the frames up to just before. If such a process is performed, the filter control signal for the current frame is not required to generate the smoothed filter control signal, so that the filter processing for the ultrasonic signal of the current frame can be completed without any additional delay from a time when the ultrasonic signal is obtained. That is, high real-time performance up to image display is maintained.

In the present embodiment, by comparing the signals before and after the filter, the evaluator 107 can grasp a signal removed as noise and directly evaluate it, thereby removing the noise so as to more closely match characteristics of the subject.

Since other configurations are the same as those in Embodiment 1, the description thereof will be omitted.

### Embodiment 5

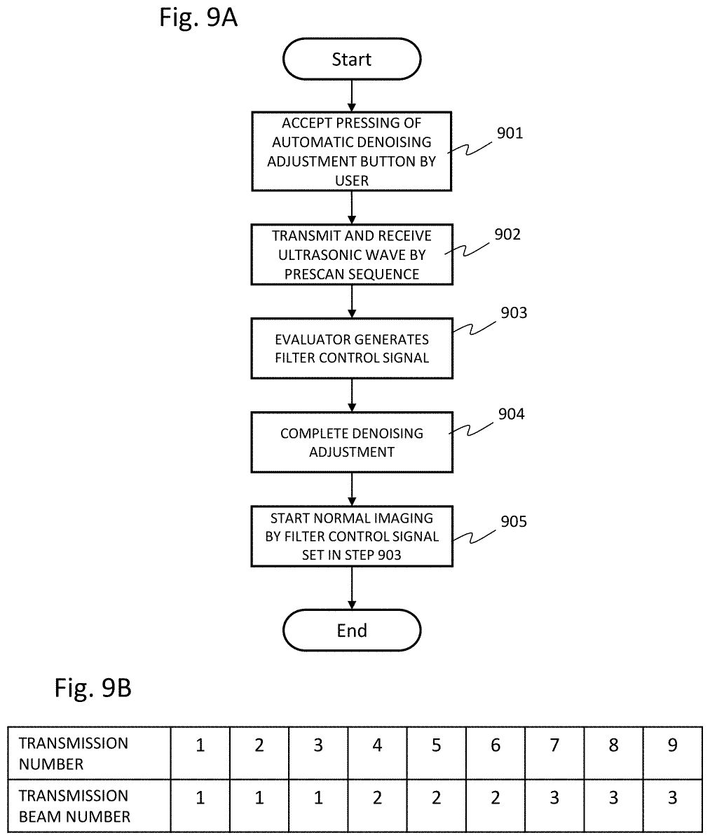

The ultrasonic imaging apparatus of Embodiment 5 will be described with reference to FIGS. 9A and 9B.

The ultrasonic imaging apparatus of Embodiment 5 is different from that of Embodiment 1 in that it transmits and receives an ultrasonic wave by a prescan sequence according to the flow of FIG. 9A before an imaging operation illustrated in the flow of FIG. 4 of Embodiment 1, to adjust the filter control signal in advance.

In the prescan sequence, as illustrated in the example of FIG. 9B, the transmission of the same transmission beam is repeated a plurality of times, a signal component and a noise component are calculated, and the filter control signal is set according to their characteristics. For example, for data received when the same transmission beam is transmitted repeatedly a plurality of times, it is possible to discriminate that the component that does not change between transmissions is the signal component, and the component that changes is the noise component. If an interval between repeated transmissions is shorter than a time it takes for an observation target to typically move by about a wavelength of the transmitted ultrasonic wave due to body movement, it is possible to discriminate the noise component including the body movement from the signal component by a method described here.

Thus, by repeatedly transmitting the same transmission beams continuously in time, it is less susceptible to influence of the body movement when estimating the noise and the signal, and it is possible to generate the filter control signal that can effectively remove the noise.

The prescan sequence will be specifically described with reference to the flow of FIG. 9A.

If the controller 92 accepts that the user has pressed an automatic denoising adjustment button disposed on the operation unit 93 of the console 103 (Step 901), the controller 92 performs the prescan sequence in which the transmission beams defined in FIG. 9B are sequentially transmitted, to generate the ultrasonic signal (reception beam) from the received signal (Step 902). The evaluator 107 generates the filter control signal by the same method as in Steps 401 to 403 of FIG. 4 (Step 903).

Thus, the controller 92 completes denoising adjustment, and uses the filter control signal set in Step 903 as an initially set filter control signal, to perform Steps 401 to 408 of the flow of FIG. 4 and generate the ultrasonic image by normal imaging (Steps 904 and 905).

In the above Step 902, as illustrated in FIG. 9B, the transmission beams having the same intensity and the same frequency characteristics (the transmission beams having the same transmission beam number) are repeatedly transmitted three times toward the same position, and the evaluator 107 generates the filter control signal 202 each time. Thus, it becomes less susceptible to the influence of the body movement of the subject, thereby generating the filter control signal that selects a filter that can effectively remove the noise. However, the number of repetitions of transmission beams and a transmission beam number and the like in the sequence shown in FIG. 9B are merely an example, and the same effect as shown in the present embodiment can be expected by arbitrarily setting of the transmission beams and the number of transmission beams.

Since other configurations are the same as those in Embodiment 1, the description thereof will be omitted.

### Embodiment 6

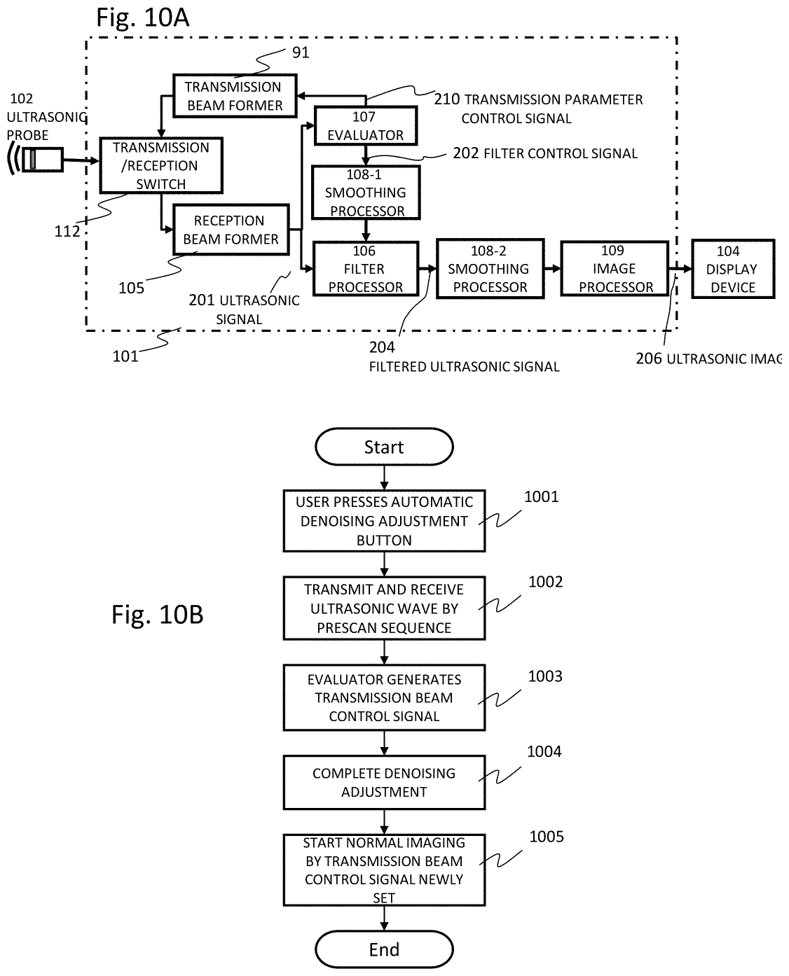

The ultrasonic imaging apparatus of Embodiment 6 will be described with reference to FIGS. 10A and 10B.

As illustrated in FIG. 10A, the ultrasonic imaging apparatus of Embodiment 6 is different from Embodiment 1 in that the evaluator 107 outputs a transmission parameter control signal 210 to the transmission beamformer 91. The transmission parameter control signal 210 is a signal for setting a parameter of the transmission beam for reducing the noise of the ultrasonic signal 201, and has been obtained by the prescan sequence.

The prescan sequence will be specifically described with reference to the flow of FIG. 10B.

When the controller 92 accepts that the user has pressed the automatic denoising adjustment button disposed on the operation unit 93 (Step 1001), the controller 92 performs the prescan sequence, transmits the transmission beam, and generates the ultrasonic signal (reception beam) from the obtained received signal (Step 1002).

The evaluator 107 determines the transmission parameter (for example, the transmission frequency) for reducing the noise of the ultrasonic signal, for example, by calculating a signal-to-noise ratio of the signal obtained by the parameters of different transmission beams and determining a transmission parameter that gives the signal-to-noise ratio not less than a certain value, and generates the transmission parameter control signal (Step 1003).

Thus, the controller 92 completes the denoising adjustment, and the evaluator 107 performs Steps 401 to 408 of the flow of FIG. 4 while outputting the transmission parameter control signal set in Step 1003 to the transmission beamformer, and generates the ultrasonic image by normal imaging (Steps 1004 and 1005).

According to the present embodiment, it is possible to transmit the transmission beam capable of reducing the noise of the ultrasonic signal, and both the signal-to-noise ratio and resolution can be achieved in a wide range due to an interaction with a noise reduction effect at the time of filtering the ultrasonic signal described in Embodiment 1.

Since other configurations are the same as those in Embodiment 1, the description thereof will be omitted.

In the prescan sequence, the transmission parameter control signal 210 may be set after separating the noise component and the signal component by continuously transmitting the same transmission beam as shown in FIG. 9B, or the transmission parameter control signal 210 may be set by continuously transmitting different transmission beams and analyzing a difference in the signals between the different transmission beams.

### Embodiment 7

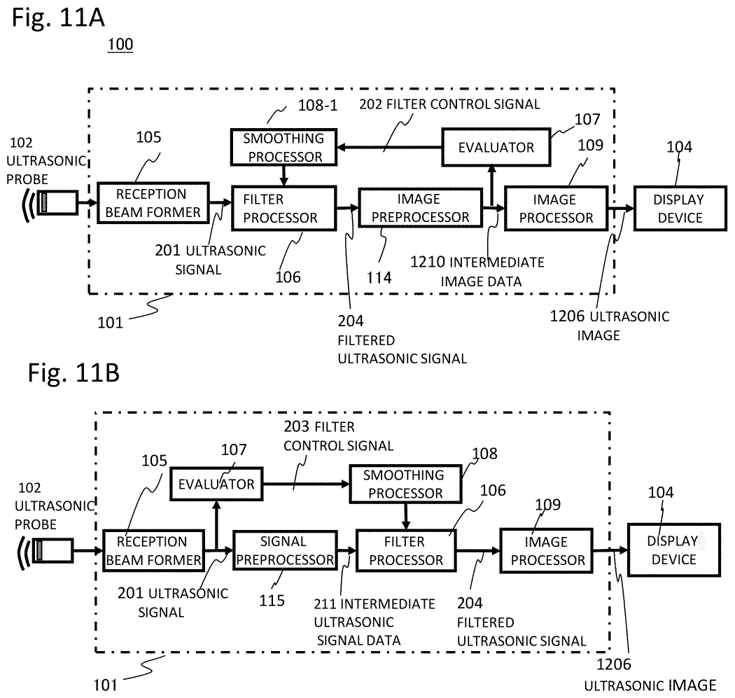

The ultrasonic imaging apparatus according to Embodiment 7 will be described with reference to FIGS. 11A and 11B.

The apparatus of FIG. 11A is different from that of Embodiment 1 in that it includes an image preprocessor 114 downstream of the filter processor 106, and inputs an intermediate image data 1210 output by the image preprocessor 114 to the evaluator 107.

The image preprocessor 114 outputs the intermediate image data 1210 by performing image preprocessing on the filtered ultrasonic signal 204. The image preprocessing includes, for example, coordinate conversion processing from the ultrasonic signal to the image, detection processing, and interpolation processing.

The evaluator 107 calculates an image characteristic value of the intermediate image data 1210, and sets the filter control signal that sets the characteristic value of the filter for removing the noise included in the ultrasonic signal based on the image characteristic value, so that the filter control signal 202 is generated and output. Examples of the image characteristic value include the frequency characteristics and the signal strength in a certain region in a certain intermediate image data 1210.

Thus, by using input signals appropriate for each of the evaluator and the filter processor, it is possible to maximize a denoising effect and minimize a calculation scale.

In FIG. 11A, only the intermediate image data 1210 is input to the evaluator 107, but the ultrasonic signal 201 is further input and the filter control signal for setting the filter characteristic value may be set based on both the image characteristic value and the signal characteristic value respectively calculated from the intermediate image data 1210 and the ultrasonic signal 201.

The apparatus of FIG. 11B is different from that of Embodiment 1 in that it includes the signal preprocessor 115 downstream of the reception beamformer 105, and the filter processor 106 processes intermediate ultrasonic signal data 211 output by the signal preprocessor 115.

The signal preprocessor 115 outputs the intermediate ultrasonic signal data 211 by performing signal processing on the ultrasonic signal 201. The signal processing here includes, for example, frequency filter processing.

The evaluator 107 calculates the signal characteristic value of the intermediate ultrasonic signal data 211, and sets the filter control signal that sets the filter characteristic value for removing the noise included in the ultrasonic signal based on the signal characteristic value, so that the filter control signal 202 is generated and output.

Thus, by using the input signals appropriate for each of the evaluator and the filter processor, it is possible to maximize the denoising effect and minimize the calculation scale.

### Embodiment 8

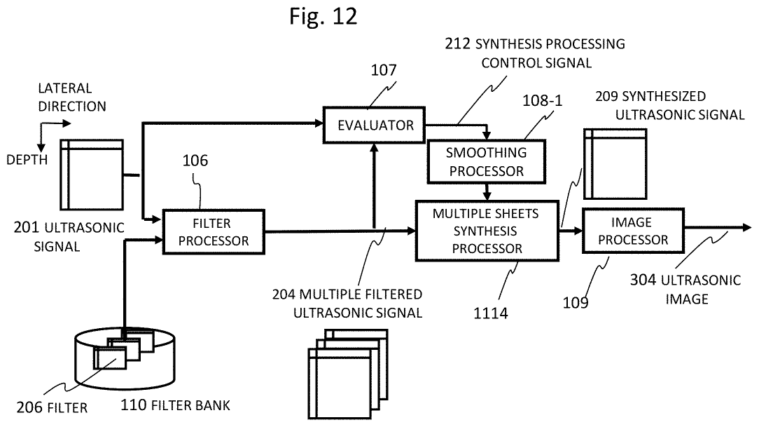

The ultrasonic imaging apparatus of Embodiment 8 will be described with reference to FIG. 12.

In the apparatus of FIG. 12, the filter processor 106 generates multiple types of filtered ultrasonic signals 204 by applying multiple types of filter processing to the same ultrasonic signal 201.

The evaluator 107 evaluates the signal characteristics of each of the multiple types of filtered ultrasonic signals 204 in the same manner as in Embodiment 1, to calculate a synthesis processing control signal 212. Specifically, the validity of the filter processing is evaluated based on the difference or correlation between the ultrasonic signal and the filtered ultrasonic signal, and the synthesis processing control signal is generated based on the evaluation value. The synthesis processing control signal is a parameter related to synthesis processing of the filtered ultrasonic signals, for example, a weight for each signal region of each filtered ultrasonic signal, and multiple sheets synthesis processor 1114 adds the filtered ultrasonic signals according to the weights.

The smoothing processor 108-1 smoothes a plurality of the filter control signals 203.

The synthesis processor 1114 is characterized by generating a synthesized ultrasonic signal 209, for example, by performing the synthesis processing by weighting and adding the filtered ultrasonic signals or the synthesis processing using wavelet transform on the filtered ultrasonic signals, based on the filter control signals.

The image processor 109 generates an ultrasonic image 304 using the synthesized ultrasonic signal 209.

As described above, in the configuration of the present embodiment, it is not necessary to perform the filter processing again based on evaluation results of the evaluator, so that the denoising effect can be reflected in the ultrasonic image without delay.

### DESCRIPTION OF REFERENCE NUMERALS AND SIGNS

91: transmission beamformer, 92: controller, 93: operation unit, 100: ultrasonic imaging apparatus, 101: ultrasonic imaging apparatus main body, 102: ultrasonic probe, 103: console, 104: display device, 105: reception beamformer, 106: filter processor, 107: evaluator, 108-1, 108-2: smoothing processor, 109: image processor, 151: subject.

What is claimed is:

1. An ultrasonic imaging apparatus comprising:
an evaluator that generates a filter control signal, in which the evaluator receives one or more time-series ultrasonic signals obtained by receiving an ultrasonic wave from a subject by an ultrasonic probe after transmitting an ultrasonic wave from the ultrasonic probe to the subject, and generates the filter control signal for setting a characteristic value of a filter for removing noise contained in the ultrasonic signal, for a time-series direction of the ultrasonic signal or for a predetermined direction or region set in a signal space of two or more dimensions in which a plurality of the ultrasonic signals are arranged;
a filter processor that removes the noise by processing the ultrasonic signal of the corresponding signal characteristic value by the filter having the characteristic value set based on the filter control signal; and
an image processor that generates an image using the ultrasonic signal from which the noise has been removed by the filter processor, wherein
a smoothing processor that smoothes distribution of the filter control signal in the direction or region and/or distribution of the ultrasonic signals after filter processing is disposed between the evaluator and the filter processor and/or between the filter processor and the image processor.

2. The ultrasonic imaging apparatus according to claim 1, wherein
the time-series ultrasonic signal is a signal in a depth direction of the subject, and
the predetermined direction set in the signal space is a direction in which frames obtained at the same position of the subject at different times are arranged.

3. The ultrasonic imaging apparatus according to claim 1, wherein the evaluator calculates distribution of signal characteristic values of the ultrasonic signal for a time-series direction of the ultrasonic signal or for a predetermined direction or region set in a signal space of two or more dimensions in which the ultrasonic signals are arranged, and generates the filter control signal for setting the characteristic value of the filter for removing the noise contained in the ultrasonic signal, for the direction or region, according to the signal characteristic value.

4. The ultrasonic imaging apparatus according to claim 1, wherein
the evaluator includes a learning model, and
the learning model has been trained by teacher data, that uses an ultrasonic signal as input data and a filter control signal suitable for removing the noise from the ultrasonic signal as output data.

5. The ultrasonic imaging apparatus according to claim 1, wherein the evaluator divides the direction or region by a predetermined length or area unit, and generates the filter control signal for each division.

6. The ultrasonic imaging apparatus according to claim 5, wherein
the evaluator sets the adjacent divisions to partially overlap each other, and
the smoothing processor smoothes the filter control signal in a portion where the divisions overlap each other and/or the ultrasonic signals after filter processing, by weighting and adding them.

7. The ultrasonic imaging apparatus according to claim 1, wherein the filter processor weights the characteristic value of the filter and performs a convolution operation with the ultrasonic signal.

8. The ultrasonic imaging apparatus according to claim 1, wherein the evaluator includes a trained learning model that uses the ultrasonic signal as an input and outputs the filter control signal.

9. The ultrasonic imaging apparatus according to claim 1, further comprising a controller that causes the evaluator to calculate an initial value of the filter control signal in advance by transmitting the ultrasonic wave to the subject by a predetermined prescan sequence and obtaining the ultrasonic signal.

10. The ultrasonic imaging apparatus according to claim 9, wherein in the prescan sequence, ultrasonic waves with the same transmission conditions are repeatedly transmitted to the subject multiple times.

11. The ultrasonic imaging apparatus according to claim 1, further comprising a transmission controller that transmits a transmission signal to the ultrasonic probe to transmit the ultrasonic wave to the subject, wherein
in addition to generating the filter control signal, the evaluator generates and outputs a transmission control signal for controlling the transmission controller, and
the transmission controller changes the ultrasonic wave transmitted from the probe by changing the transmission signal based on the transmission control signal.

12. The ultrasonic imaging apparatus according to claim 11, wherein the evaluator transmits the ultrasonic wave to the subject by a predetermined prescan sequence, obtains the ultrasonic signal, and calculates ultrasonic transmission parameters for reducing the noise.

13. The ultrasonic imaging apparatus according to claim 1, further comprising a synthesis processor that weights and synthesizes the ultrasonic signal from which the noise has been removed by the filter processor and the ultrasonic signal before the noise is removed by the filter processor, wherein the image processor generates the image using the ultrasonic signal synthesized by the synthesis processor.

14. The ultrasonic imaging apparatus according to claim 13, further comprising an operation unit that receives weights used for weighting by the synthesis processor from a user.

15. An image processing apparatus comprising:
an evaluator that generates a filter control signal, in which the evaluator receives one or more time-series ultrasonic signals obtained by receiving an ultrasonic wave from the subject by the ultrasonic probe, and generates the filter control signal for setting a characteristic value of a filter for removing noise contained in the ultrasonic signal, for a time-series direction of the ultrasonic signal or for a predetermined direction or region set in a signal space of two or more dimensions in which a plurality of the ultrasonic signals are arranged;
a filter processor that removes the noise by processing the ultrasonic signal of the corresponding signal characteristic value by the filter having the characteristic value set based on the filter control signal; and
an image processor that generates an image using the ultrasonic signal from which the noise has been removed by the filter processor, wherein
a smoothing processor that smoothes distribution of the filter control signal in the direction or region and/or distribution of the ultrasonic signals after filter processing is disposed between the evaluator and the filter processor and/or between the filter processor and the image processor.
